(19) United States
(12) Patent Application Publication
(10) Pub. No.: US 2010/0093012 A1
(43) Pub. Date: Apr. 15, 2010

(54) DIAGNOSIS AND RISK STRATIFICATION OF CARDIAC INSUFFICIENCY USING NEUROPHYSIN

(75) Inventors: Andreas Bergmann, Berlin (DE); Joachim Struck, Berlin (DE)

Correspondence Address: CONNOLLY BOVE LODGE & HUTZ, LLP, P O BOX 2207, WILMINGTON, DE, 19899, US

(73) Assignee: BRAHMS Aktiengesellschaft, Henningsdorf (DE)

(21) Appl. No.: 12/518,180

(22) PCT Filed: Dec. 10, 2007
(86) PCT No.: PCT/DE2007/002215

§ 371 Date: Dec. 22, 2009

(30) Foreign Application Priority Data
Dec. 8, 2006 (DE) 10 2006 058 266.7

(51) Int. Cl. C12Q 1/28; C12Q 1/02; C12Q 1/25

(52) U.S. Cl. 435/28; 435/29

(57) ABSTRACT

The invention relates to a method for the diagnosis and/or risk stratification of cardiac insufficiency, according to which the marker neurophysin or a fragment or partial peptide thereof is determined on patients to be examined. The invention also relates to advantageous marker combinations containing neurophysin, and to a diagnostic device and a kit for carrying out the method.

The invention relates to a method for the diagnosis and/or risk stratification of cardiac insufficiency, wherein a determination of the marker neurophysin or a fragment or partial peptide thereof is carried out on a patient to be examined. The invention relates to advantageous marker combinations containing neurophysin. Furthermore, the invention relates to a diagnostic device and a kit for carrying out the method.

In Europe about one million patients a year arrive in hospital emergency rooms with the symptom of acute respiratory distress. Respiratory distress is a characteristic symptom of many diseases and can be attributed to cardiac insufficiency in approximately 35-47% of the cases (Januzzi J L Jr., Camargo C A, Anwaruddin S., Baggish A L, Chen A A, Krauser D G, Tung R, Cameron R, Nagurney J T, Chae C U, Lloyd-Jones D M, Brown D F, Foran-Melanson S, Sluss P M, Lee-Lewandrowski E, Lewandrowski K B, The N-terminal Pro-BNP investigation of dyspnea in the emergency department (PRIDE) study, Am J Cardiol. 95 (8) (2005), pp. 948-954 and Maisel A S, Krishnaswamy P, Nowak R M, McCord J, Hollander J E, Duc P, Omland T, Storrow A B, Abraham W T, Wu A H, Clopton P, Steg P G, Westheim A, Knudsen C W, Perez A, Kazanegra R, Herrmann H C, McCullough P A; Breathing Not Properly Multinational Study Investigators, Rapid measurement of B-type natriuretic peptide in the emergency diagnosis of heart failure, N Engl J Med. 347 (3) (2002), pp. 161-167).

In the early stages the patient often notices the cardiac insufficiency only a little. Untreated, the disease as a rule increases in severity and in the late stage leads to complete physical exhaustion even at rest. The lack of nourishment of all of the body's organs, including the cardiac itself can lead to death at this stage. Once the disease is advanced, life expectancy is considerably reduced even with optimum therapy (approx. 30% of deaths per year). It is therefore important to recognize a cardiac insufficiency as early as possible and to deal with its causes consistently.

In order to start a suitable therapy, an early diagnosis and differentiation of the underlying disease is therefore necessary at an early stage and in emergency and intensive medicine. Both the differentiation and delimitation of cardiac insufficiency from other diseases are often made difficult by unspecific symptoms (respiratory distress, coughing).

A test is available using the determination of the plasma concentration of the brain natriuretic peptide (BNP or NTproBNP), which is also used in the course of daily routine for the diagnosis of cardiac insufficiency (Maisel et al. (supra)).

In the prior art neurophysin is described as a marker for nicotine absorption (Robinson A G. Isolation, assay and secretion of individual human neurophysins. J Clin Invest 1975; 55: 360-7), cancer and non-cancer associated SIADH (Syndrome of inappropriate ADH secretion) and nephrogenic diabetes insipidus (Pullan P T, Clappison B H, Johnston C I. Plasma vasopressin and human neurophysins in physiological and pathological states associated with changes in vasopressin secretion. J Clin Endocrinol Metab 1979; 49; 580-7; North W G, LaRochelle F T, Jr., Melton J, Mills R C. Isolation and partial characterization of two human neurophysins: their use in the development of specific radioimmunoassays. J Clin Endocrinol Metab 1980; 51: 884-91).

In the prior art there is a great need to identify and provide further markers for the indication of cardiac insufficiency and to supply them to clinical practice.

It is therefore the object of the present invention to provide a method for the diagnosis and/or risk stratification of cardiac insufficiency.

However, the disadvantage of known diagnostic methods using the markers previously known is that an early and complete detection of risk patients is not successful and therefore a risk stratification is carried out only to an unsatisfactory extent. One further object on which the invention is based therefore lies in developing a method for the risk stratification of cardiac insufficiency, which renders possible an improved detection of risk patients.

The object is attained through a method for the diagnosis and/or risk stratification of cardiac insufficiency, wherein a determination of the marker neurophysin or a fragment or partial peptide thereof is carried out on a patient to be examined (hereinafter referred to as the method according to the invention).

Within the scope of this invention “cardiac insufficiency” is understood to mean an acute or chronic inability of the heart to supply tissue with sufficient blood and as a result thereof with sufficient oxygen, in order to ensure tissue metabolism at rest or under stress. Clinically, a cardiac insufficiency is present when typical symptoms (dyspnea, fatigue, liquid retention) exist which are based in origin on a cardiac functional disorder in terms of a systolic or diastolic functional disorder. Chronic cardiac insufficiency (CHF) is likewise covered according to the invention (Kardiologie compact, edited by Chrisian Mewis, Reimer Riessen and Ioakim Spyridopouolos, 2^{nd} unamended edition, Thieme 2006). The causes of a cardiac insufficiency can be: cardiac valve defect (e.g., as the late symptom of rheumatic fever), myocarditis (inflammation of the myocardium), cardiac arrhythmias, cardiac infarction together with excessively high blood pressure (hypertonia) and/or arteriosclerosis (calcification) of the coronary vessels (coronary heart disease). Furthermore, the following are covered according to the invention: hypertensive heart disease with (congestive) cardiac insufficiency, hypertensive heart disease and kidney disease with (congestive) cardiac insufficiency, primary dextrocardiac insufficiency, secondary dextrocardiac insufficiency, left ventricular insufficiency without symptoms (NYHA stage I), left ventricular insufficiency with symptoms with greater stress (NYHA stage II), left ventricular insufficiency with symptoms with slight stress (NYHA stage III), left ventricular insufficiency with symptoms at rest (NYHA stage IV) and cardiogenic shock.

The method according to the invention therefore likewise covers the above-referenced indications. Furthermore, all of the cited indications are described, e.g., in Pschyrembel, De Gruyter, Berlin 2004.

According to the invention, the term “risk stratification” covers discovering patients, in particular emergency patients and risk patients, with the worse prognosis for the purpose of more intensive diagnosis and therapy/treatment of cardiac insufficiency with the objective of rendering possible the most favorable possible progress. A risk stratification according to the invention consequently allows an effective treatment method, which are given with respect to a cardiac insufficiency.

Particularly advantageously, in particular in cases of emergency and/or intensive care medicine, a reliable diagnosis can be made by means of the method according to the invention. The method according to the invention renders possible clinical decisions that lead to a rapid therapeutic success. Clinical decisions of this type likewise include further treatment by means of pharmaceuticals for the treatment or therapy of cardiac insufficiency, such as ACE inhibitors, AT1 antagonists: blockers of the angiotensin II receptor (subtype I), beta blockers bisoprolol, carvedilol, metoprolol and nebivolol, vasopressin receptor antagonists, aldosterone antagonists from NYHA stage III, calcium sensitizers (Levosimendan).

In a further preferred embodiment the method according to the invention therefore relates to the therapeutic control of a cardiac insufficiency.

The invention therefore likewise relates to a method for the risk stratification of patients, in particular for the stratification of patients for clinical decisions, preferably in time-critical intensive medicine or emergency medicine and for the hospitalization of patients

In a further preferred embodiment of the method according to the invention the diagnosis is carried out for prognosis, for the differential diagnostic early detection and detection, for the assessment of the degree of severity and for the assessment of the course of a cardiac insufficiency accompanying therapy.

In a further embodiment of the method according to the invention body fluid, in particular blood, optionally whole blood or serum, is taken from the patient to be examined and the diagnosis is carried out in vitro/ex vivo, i.e., outside the human or animal body. The diagnosis can be carried out based on the determination of the marker neurophysin and the quantity thereof present in at least one patient sample.

Within the scope of this invention “neurophysin” (or neurophysin II) (fragment: AS32-124 of the preprovasopressin; see FIG. 2) is understood to be a polypeptide/protein containing free 93 amino acid (93AS: SEQ ID no. 1: AMSDLELRQC LPCGPGGKGR CFGPSICCAD ELGCFVGTAE ALRCQEENYL PSPCQSGQKA CGSGGRCAAF GVCCNDESCV TEPECREGFH RRA) or fragments or partial peptides thereof. Furthermore, this polypeptide according to the invention can have posttranslational modifications, such as glycolization, lip(o)idization or derivatizations. Neurophysin is surprisingly stable in plasma.

In a further embodiment the determination of neurophysin can additionally be made with further markers, namely preferably those that already indicate a cardiac insufficiency and permit a synergistic effect of marker combinations containing neurophysin in the method according to the invention.

The invention therefore relates to such an embodiment of the method according to the invention, wherein the determination is additionally carried out on a patient to be examined with at least one further marker selected from the group of inflammatory markers, cardiovascular markers, neurohormonal markers or ischemic markers.

According to the invention, the inflammatory marker can be selected from at least one marker from the group of C-reactive protein (CRP), cytokinin, such as, for example, TNF-alpha, interleukins, such as, for example, IL-6, procalcitonin (1-116, 3-116) and adhesins, such as VCAM or ICAM, and the cardiovascular marker from at least one marker from the group creatincinase, myeloperoxidase, copeptin, myoglobin, natriuretic protein, in particular ANP (or ANF), proANP, NT-proANP, BNP, proBNP, NT-proBNP or respectively a partial sequence thereof, cardial troponin, CRP. Furthermore, these are likewise understood to include (pro)hormones regulating the circulation, in particular like pro-gastrin-releasing peptide (proGRP), pro-endothelin (proEnd), pro-leptin, pro-neuropeptide-Y, pro-somatostatin, pro-neuropeptide-YY, pro-opiomelanocortin, pro-adrenomedullin (proADM), copeptin or respectively a partial sequence thereof.

The ischemic marker can be selected from at least one marker from the group troponin and T, CK-MB. Furthermore, the neurohormonal marker can be at least one natriuretic protein, in particular ANP (or ANF), proANP, NT-proANP, BNP, proBNP, NT-proBNP or respectively a partial sequence thereof.

Particularly preferred are marker combinations of neurophysin with a prohormone, in particular copeptin, and/or BNP, proBNP, NT-proBNP.

In a further embodiment of the invention the method according to the invention can be carried out by means of parallel or simultaneous determinations of the markers (e.g., multititration plates with 96 and more cavities), wherein the determinations are carried out on at least one patient sample.

Furthermore, the method according to the invention and the determinations thereof can be carried out on an automatic analyzer, in particular by means of a Kryptor (http://www.kryptor.net/).

In another embodiment, the method according to the invention and the determinations thereof can be carried out by means of a rapid test (e.g., a lateral-flow test), whether in single-parameter or multiple-parameter determination.

Furthermore, the invention relates to the use of neurophysin or a fragment or partial peptide thereof and optionally further markers, as listed above, for the diagnosis and/or risk stratification of cardiac insufficiency.

Another object is the provision of a corresponding diagnostic device or the use thereof for carrying out the method according to the invention.

Within the scope of this invention, a diagnostic device in particular an array or assay (e.g., immunoassay, ELISA, etc.) is understood to be in the broadest sense a device for carrying out the method according to the invention.

The invention furthermore relates to a kit for the diagnosis or risk stratification of cardiac insufficiency, containing analytical reagents for determining the marker neurophysin or a fragment or partial peptide thereof and optionally the markers listed above. Analytical reagents of this type comprise, e.g., antibodies, antibody fluorescence, etc.

The following examples and figures are used to explain the invention in more detail, but without restricting the invention to these examples and figures.

### EXAMPLES AND FIGURES

A blood sample was taken from patients who reported to the emergency room of a hospital with the indicating symptom of respiratory distress during the initial examination. EDTA plasma obtained through centrifugation was aliquoted and stored at −80° C. until the measurement of neurophysin. According to earlier descriptions (Pullan (supra)) a radioimmunoassay was developed for neurophysin: neurohypophyseal neurophysin was isolated and quantified. Rabbits were immunized therewith and thus high-titered anti-neurophysin antisera obtained. For the immunoassay the highest-titered antiserum was used in a concentration of 1:100,000. Purified neurophysin was radio-iodized with the chloramines T method and used as a tracer in the assay. Dilutions of purified neurophysin in normal horse serum were used as standards. The assay was carried out as follows: 50 μl sample or standard was mixed with 100 μl tracer (12,000 dpm per determination) and 100 μl diluted anti-neurophysin antiserum and incubated for 24 hours at 4° C. 100 mM sodium phosphate, pH 7.5, 0.1% BSA was used as a buffer. Antibody-bound tracer was separated from free tracer in that 60% ethanol was added and then centrifuged for 15 minutes at 4° C. and 5,000 g. The supernatant was discarded and the radioactivity remaining in the pellet was determined. The evaluation was carried out with the aid of Multicalc software. The assay had an analytical detection limit of 22 pg/ml and a measuring range up to 400 pg/ml. Plasma samples from different patients, as explained below, were measured with the assay. Samples with measured values>400 pg/ml were measured in suitable dilutions, so that measured values within the measuring range were obtained.

The diagnosis of cardiac insufficiency with the patients examined was based on the Framingham Score (McKee P A, Castelli W P, McNamara P and Kannel W B, The natural history of congestive heart failure: the Framingham study, N Engl J Med 285 (1971), pp. 1441-1446) for cardiac insufficiency plus echographic evidence of a systolic or diastolic dysfunction.

Furthermore, blood was taken from healthy individuals without known disease and EDTA plasma was obtained through centrifugation.

Clinical Utility

Normal Range

Neurophysin concentrations were determined in samples from healthy control subjects (n=20). The median was 70.3 pg/ml, the lowest measured value was 8.8, the highest 220 pg/ml, the 95% percentiles 36.5 or 135 pg/ml.

Cardiac Insufficiency/Degree of Severity

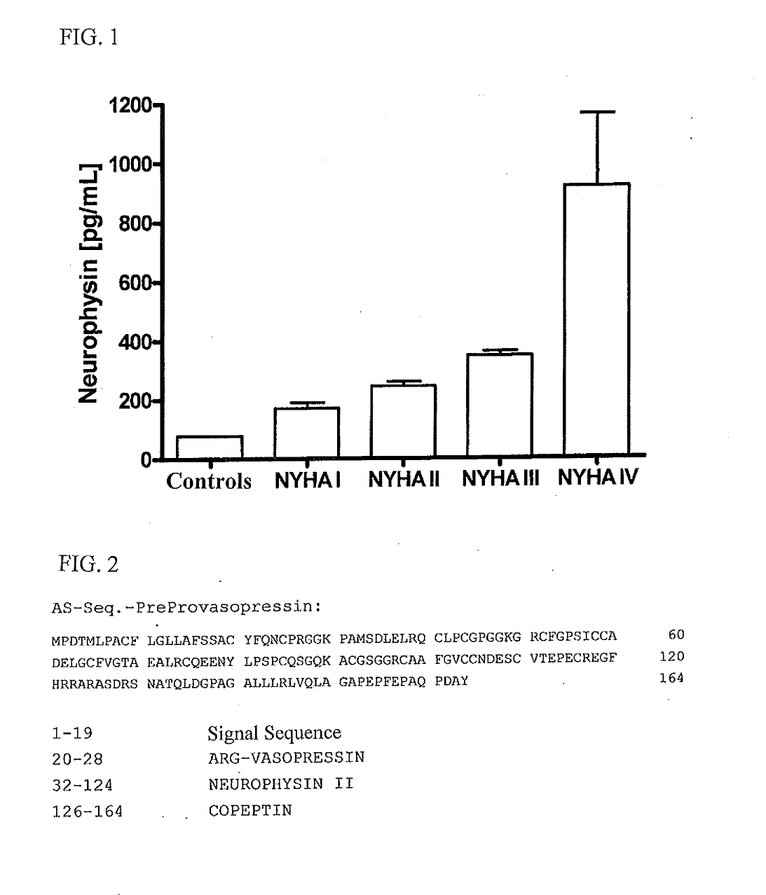

Neurophysin concentrations were measured in patients with chronic or acute decompensated cardiac insufficiency. Neurophysin concentrations were associated with the degree of severity of the cardiac insufficiency: the average values of the neurophysin concentrations in the four NYHA categories of degrees of severity I-IV were: 171.4, 243.4, 346.9 and 918.1 pg/ml respectively (see FIG. 1).

Chronic Cardiac Insufficiency/Diagnosis

Neurophysin values were determined from a group of 258 patients with chronic cardiac insufficiency and 200 healthy controls. The receiver operator characteristics analysis yielded an AUC of 0.89. With a cut-off value of 213 pg/ml, a sensitivity of 48% resulted with a specificity of 98%. With a cut-off value of 136.1 pg/ml, a sensitivity of 68.2% resulted with a specificity of 95%.

Chronic Cardiac Insufficiency/Prognosis

Neurophysin values were determined from a group of 258 patients with chronic cardiac insufficiency. The patients were observed over an average period of 360 days. Within this period 80 patients died, 178 survived. The best cut-off value (defined as the greatest product from sensitivity and specificity) for the prognosis of mortality was determined through receiver operator characteristics analysis: 247 pg/ml. With this cut-off value the sensitivity of the prognosis was 59.5%, the specificity was 65.2%. The likelihood ratio with a cut-off value of 247 pg/ml was 1.7 (see table below).

| | <247 pg/ml | >247 pg/ml |
|---|---|---|
| Survived | 116 | 62 |
| Died | 32 | 47 |

Acute Cardiac Insufficiency/Diagnosis

Neurophysin values were determined from a group of 125 patients with acute respiratory distress. 69 patients out of the 125 patients had cardiac insufficiency. The receiver operator characteristics analysis for the differential diagnosis of the cardiac insufficiency yielded an AUC of 0.61. With a cut-off value of 4940 pg/ml, a sensitivity of 6.6% resulted with a specificity of 98%. With a cut-off value of 3000 pg/ml, a sensitivity of 11.7% resulted with a specificity of 95%.

Acute Cardiac Insufficiency/Prognosis

Neurophysin values were determined from a group of 69 patients with acute decompensated cardiac insufficiency. The patients were observed over a period of 360 days. Within this period 21 patients died, 48 survived. The best cut-off value (defined as the greatest product of sensitivity and specificity) was determined for the prognosis of mortality through receiver operator characteristics analysis: 885 pg/ml. With this cut-off value the sensitivity of the prognosis was 57.1%, the specificity was 75%. The likelihood ratio with a cut-off value of 885 pg/ml was 2.3 (see table below).

| | <885 pg/ml | >885 pg/ml |
|---|---|---|
| Survived | 36 | 12 |
| Died | 9 | 12 |

TABLE
Classification of the New York Heart Association (NYHA)

| | |
|---|---|
| NYHA I | No limitation of physical activity. Ordinary physical activity |
| | does not cause undue fatigue, palpitation, dyspnea (shortness |
| | of breath), or angina pectoris. |
| NYHA II | Slight limitation of physical activity. Comfortable at rest, but |
| | ordinary physical activity results in fatigue, palpitation, |
| | dyspnea or angina pectoris. |
| NYHA III | Marked limitation of physical activity. Comfortable at rest, |
| | but less than ordinary activity causes fatigue, palpitation, |
| | dyspnea or angina pectoris . . . |
| NYHA IV | Symptoms of cardiac insufficiency with any physical activity |
| | and at rest. Confined to bed. |

1. Method for the diagnosis and/or risk stratification of cardiac insufficiency, characterized in that a determination of the marker neurophysin or a fragment or partial peptide thereof is carried out on a patient to be examined.

2. Method according to claim 1, characterized in that the method is an in-vitro diagnosis.

3. Method according to claim 1, characterized in that the cardiac insufficiency covers a chronic cardiac insufficiency, hypertensive heart disease with (congestive) cardiac insufficiency, hypertensive heart disease and kidney disease with (congestive) cardiac insufficiency, primary dextrocardiac insufficiency, secondary dextrocardiac insufficiency, left ventricular insufficiency without symptoms (NYHA stage I), left ventricular insufficiency with symptoms with greater stress (NYHA stage II), left ventricular insufficiency with symptoms with slight stress (NYHA stage III), left ventricular insufficiency with symptoms at rest (NYHA stage IV), cardiogenic shock, myocarditis, cardiac arrhythmias and/or hypertonia.

4. Method for the diagnosis and/or risk stratification of cardiac insufficiency according to claim 1 for carrying out clinical decisions, in particular further treatment and therapy by means of pharmaceuticals, in particular in intensive care medicine or emergency medicine and for the hospitalization of the patient.

5. Method for the diagnosis of cardiac insufficiency according to claim 1 for the prognosis, for differential diagnostic early detection and detection, for the assessment of the degree of severity and for the assessment of the course of a cardiac insufficiency accompanying therapy.

6. Method according to claim 1, characterized in that in addition a determination is carried out on a patient to be examined with at least one further marker selected from the group of inflammatory markers, cardiovascular markers, neurohormonal markers or ischemic markers.

7. Method according to claim 1, characterized in that the inflammatory marker is selected from at least one marker from the group of C-reactive protein (CRP), cytokinin, such as, for example, TNF-alpha, interleukins, such as, for example, IL-6, procalcitonin (1-116, 3-116) and adhesins, such as VCAM or ICAM.

8. Method according to claim 1, characterized in that the cardiovascular marker is selected from at least one marker from the group creatincinase, myeloperoxidase, copeptin, myoglobin, natriuretic protein, in particular ANP (or ANF), proANP, NT-proANP, BNP, proBNP, NT-proBNP or respectively a partial sequence thereof, cardial troponin, CRP and (pro)hormones regulating the circulation, like pro-gastrin-releasing peptide (proGRP), pro-endothelin-1, pro-leptin, pro-neuropeptide-Y, pro-somatostatin, pro-neuropeptide-YY, pro-opiomelanocortin, pro-adrenomedullin (proADM), copeptin or respectively a partial sequence thereof.

9. Method according to claim 1, characterized in that the ischemic marker is selected from at least one marker from the group troponin I and T, CK-MB.

10. Method according to claim 1, characterized in that the neurohormonal marker is at least one natriuretic protein, in particular ANP (or ANF), proANP, NT-proANP, BNP, proBNP, NT-proBNP or a partial sequence thereof.

11. Method according to claim 1, characterized in that parallel or simultaneous determinations of the markers are carried out.

12. Method according to claim 1, characterized in that the determinations are carried out on at least one patient sample.

13. Method according to claim 1, characterized in that the determinations are carried out on an automatic analyzer, in particular by means of a Kryptor.

14. Method according to claim 1, characterized in that the determinations are carried out by means of a rapid test, in particular in single-parameter or multiple-parameter determinations.

15. (canceled)

16. Diagnostic for carrying out a method according to claim 1.

17. Kit for the diagnosis and/or risk stratification of cardiac insufficiency, containing analytical reagents for determining the marker neurophysin or a fragment or partial peptide thereof and optionally further markers according to claim 6 and auxiliaries.


SEQUENCE LISTING (2 sequences)

SEQ ID NO: 1 - protein, 93 aa, Homo sapiens
AMSDLELRQCLPCGPGGKGRCFGPSICCADELGCFVGTAEALRCQEENYLPSPCQSGQKACGSGGRCAAFGVCCNDESCVTEPECREGFHRRA

SEQ ID NO: 2 - protein, 164 aa, Homo sapiens
MPDTMLPACFLGLLAFSSACYFQNCPRGGKPAMSDLELRQCLPCGPGGKGRCFGPSICCADELGCFVGTAEALRCQEENYLPSPCQSGQKACGSGGRCAAFGVCCNDESCVTEPECREGFHRRARASDRSNATQLDGPAGALLLRLVQLAGAPEPFEPAQPDAY